(19) United States
(12) Patent Application Publication
(10) Pub. No.: US 2006/0264905 A1
(43) Pub. Date: Nov. 23, 2006

(54) Improved Catheters

(75) Inventors: Joseph Eskridge, Clyde Hill, WA (US); Gilbert Clarke, Seattle, WA (US)

Correspondence Address: SPECKMAN LAW GROUP PLLC, 1201 THIRD AVENUE, SUITE 330, SEATTLE, WA, 98101, US

(73) Assignee: Pulsar Vascular, Inc., Seattle, WA (US)

(21) Appl. No.: 11/224,372

(22) Filed: Sep. 12, 2005

Related U.S. Application Data
(60) Provisional application No. 60/676,925, filed on May 2, 2005.

(51) Int. Cl. A61M 25/00

(52) U.S. Cl. 604/523

(57) ABSTRACT

Catheters having a tapered, atraumatic distal tip are provided. In one embodiment, the outer surface of the distal portion of the catheter has a generally non-cylindrical and substantially triangular cross-sectional configuration. The inner surface cross-sectional configuration of the distal portion of the catheter may match the outer surface, or it may have a cylindrical or oval configuration. The terminal orthogonal surface of the distal catheter tip is chamfered or rounded or contoured. In another embodiment, inner and/or outer catheter surfaces have a three dimensional surface conformation and may be dimpled or grooved. The grooves may be generally liner or curved or helical or in a spiral configuration. Dimpled and/or grooved surface discontinuities may be provided in connection with and in addition to lubricious coatings, surfaces, and the like.

### CROSS-REFERENCE TO RELATED APPLICATION

This application claims priority under 35 U.S.C. 119(e) to U.S. Patent Application No. 60/676,925, filed May 2, 2005.

### TECHNICAL FIELD OF THE INVENTION

The present invention relates to improved catheters having atraumatic distal conformations and surface discontinuities that ease guidance through vessels or other lumens or cavities and prevent damage to vessel walls, as well as improving operation in connection with associated devices and procedures.

### BACKGROUND OF THE INVENTION AND DESCRIPTION OF PRIOR ART

Catheters are flexible tubes used for navigating internal body vessels and lumens and guiding devices within the body, such as in the vasculature, spinal column, fallopian tubes, bile ducts, and the like, and are often used in connection with minimally invasive diagnostic or surgical techniques. Catheters may be used for medical procedures to examine, diagnose and treat internal conditions while positioned at a specific location within the body that is otherwise inaccessible. An intravascular catheter is generally inserted and advanced through a valved introducer fitting into a blood vessel near the surface of the body, such as the femoral artery, and is guided through the vasculature to a desired location. Catheters are used for coronary vascular and cardiac-related interventional operations, as well as neurovascular interventions, peripheral vascular, renal, and other types of intravascular interventions. Medical devices and instruments may be guided, through the catheter, to the desired site and operated.

Guiding catheters are generally the catheters through which various interventional devices and instruments are supported and guided during passage to a desired internal location. Guide catheters are sold in a variety of pre-formed sizes and shapes, customized for desired procedures. Microcatheters are generally smaller diameter catheters used for delivery of agents, devices or instruments through small diameter vessels in neurovascular interventions. Therapeutic devices and agents such as embolization coils, pharmaceutical agents, and embolic materials are delivered to a neurovascular site through microcatheters, for example. Both flow-guided and wire-guided microcatheters are used for interventional navigation. Catheters intended for use in small, delicate or tortuous vessels often have soft, shaped distal tips intended to facilitate navigation to and retention in particular target sites.

Intravascular catheters must be flexible enough to navigate through the sometimes tortuous vasculature without damaging tissue, yet stiff enough to provide “pushability” through the vasculature and support for internally guided medical devices, fluids, and the like and must be kink-resistant. Guide catheters may have a composite construction that provides greater stiffness and support in proximal areas with more softness and flexibility in distal areas. The inner lumens of guide catheters may be coated with a lubricious coating or constructed from a lubricious material such as PTFE (Teflon). The outer catheter surface may be provided with a hydrophilic coating to enhance lubricity and facilitate passage through vessels during navigation of the catheter. Microcatheters are generally smaller diameter, have a low distal profile and are soft and flexible, with a smooth inner lumen surface.

One of the problems encountered with intravascular catheters is that contact of the distal catheter tip with a vessel wall during navigation to a desired site or with a site during an intervention may injure the vascular wall or tissue at the site. Contact between a guiding catheter and a vessel wall, for example, may produce vessel dissection and/or vasospasm, which can cause complications such as clot formation or thrombosis during an intravascular procedure. Catheters having soft and/or flexible distal portions are less prone to damage vessel walls and tissue, but tissue damage and vasospasm during catheter navigation and placement remain problematic.

Yet another challenge encountered with intravascular catheters is providing quick and reliable passage of instruments and devices through the length of the catheter for delivery to a desired internal site. Inner walls of delivery catheters may comprise a lubricious layer or coating to facilitate sliding of an accessory device or instrument within the lumen, but such a coating may not provide sufficient lubricity to translate a device through the length of the catheter lumen, particularly if the path involves tortuous passages or the lumen is narrow compared to the dimensions of the accessory device or instrument.

### SUMMARY OF THE INVENTION

In one aspect, the present invention relates to catheters having a distal tip conformation that is tapered and substantially atraumatic. The distal catheter portion has a lumen that is generally axially aligned with and forms an extension of the proximal and middle section catheter lumens. In one embodiment, proximal and middle portions of a catheter have substantially cylindrical inner and outer cross-sectional conformations, while a distal portion of the catheter has an outer surface having a substantially non-cylindrical cross-sectional configuration. The distal portion of the catheter may also be tapered along a curved or linear profile and have a reduced dimension outer perimeter compared to the outer perimeter dimension(s) of the remainder of the catheter.

According to one embodiment, the outer surface cross-sectional configuration of the distal portion of the catheter is generally triangular and has arced corners separated by generally linear or slightly curved side walls. In another exemplary embodiment, the distal portion of the catheter has a generally pentagonal or hexagonal cross-sectional profile with arced corners separated by generally linear or slightly curved side walls. The material forming the distal portion of the catheter may have different, generally more flexible and resilient properties, than those of the material forming the remainder of the catheter. The arced corners may have different flexibility or elasticity properties than those of the side walls, and may have a different cross-sectional thickness than that of the side walls.

The inner surface cross-sectional configuration of the distal portion of the catheter may substantially match the configuration of the outer surface, or it may have a different configuration. In one embodiment, for example, the cross-sectional configuration of the inner surface of the distal portion forming the lumen may be generally cylindrical or oval, while the outer distal portion surface may be generally triangular. The catheter lumen may have constant diameter along the length of the catheter, including the distal portion, or the lumen at the distal catheter portion may have a smaller diameter compared to that of the proximal and/or middle catheter portions. The terminal orthogonal surface of the distal portion of the catheter is preferably chamfered or rounded or otherwise contoured to further render the distal tip atraumatic and to minimize the incidence of damage to vessel walls or other lumen surfaces.

The atraumatic catheter distal portion may be constructed integrally with the remainder of the catheter, or the atraumatic distal catheter portion may be constructed separately and installed or mounted on the remainder of the catheter. The catheter distal portion may have enhanced flexibility or elasticity compared to the flexibility or elasticity of the remainder of the catheter. The distal catheter portion may have a generally linear, angled or curved axial alignment.

According to another aspect of the present invention, inner and/or outer catheter surfaces may have a three-dimensional surface conformation. In one embodiment, for example, catheter surfaces, and particularly inner catheter surfaces forming lumens, may be dimpled or grooved or provided with other types of surface discontinuities to reduce friction and enhance the slidability of and passage through the lumen of instruments, accessory devices, and the like. The dimpled and/or grooved surface discontinuities may be provided in a regular or irregular pattern and may be provided in connection with and in addition to lubricious coatings and surfaces. In another embodiment, outer catheter surfaces may additionally or alternatively be dimpled or grooved or provided with other types of surface discontinuities.

Numerous catheter materials and constructions are known, and catheters of the present invention may be constructed using any materials, composite arrangements and conformations and construction techniques known in the art. Many catheters, for example, have a multi-layer construction and may be reinforced in sections or along their length, and may have different properties and dimensions along their length. Inner and/or outer surfaces may be provided with coatings or constructed from materials that enhance lubricity. Suitable coatings and materials are well known in the art. Radio-opaque markers may be incorporated in the catheters to allow for visualization and precise positioning, as is known in the art.

### BRIEF DESCRIPTION OF THE DRAWINGS

Various aspects of applicants' claimed inventions are illustrated schematically in the accompanying drawings, which are intended for illustrative purposes only and are not drawn to scale.

FIG. 1 shows an enlarged side view of one embodiment of a catheter distal portion of the present invention having a tapered configuration, with lines indicating the curved and tapered features of the catheter.

FIG. 2 shows an enlarged distal end side perspective view of a distal portion of a catheter of the present invention having a, tapered, generally triangular configuration.

FIG. 3 shows an enlarged distal end side perspective view of the distal portion of a catheter of the present invention having a tapered, generally hexagonal external configuration.

FIG. 4 shows an enlarged distal end side perspective view of yet another embodiment of a distal portion of a catheter of the present invention having a tapered, generally triangular external configuration, with dashed lines depicting the lumen of the distal catheter portion.

FIG. 5A shows an enlarged cross-sectional side view of one tip configuration of the present invention.

FIG. 5B shows an enlarged cross-sectional side view of another tip configuration of the present invention.

FIG. 6 shows an enlarged cross-sectional side view of a catheter of the present invention having surface discontinuities in the form of dimples on its inner lumen surface.

FIG. 7 shows an enlarged cross-sectional side view of a catheter of the present invention having surface discontinuities in the form of dimples on its outer surface.

FIG. 8 shows an enlarged cross-sectional side view of a catheter of the present invention having surface discontinuities in the form of a substantially helical groove on its inner surface.

FIG. 9 shows an enlarged end perspective view of a catheter of the present invention having surface discontinuities in the form of alternating lands and grooves on its inner lumen surface.

### DETAILED DESCRIPTION OF THE INVENTION

Catheters of the present invention comprise generally tubular structures having a substantially continuous side wall forming a lumen and may be used for a variety of purposes. Such catheters may be employed, for example, as guide catheters or delivery catheters or microcatheters for delivery of accessory devices, instruments, pharmaceuticals or other agents, or the like, to a target site within the body that is generally accessible through the vasculature or a body opening or lumen. Catheters of the present invention thus include guide and delivery catheters used for any intravascular purpose and microcatheters designed for neurovascular interventions. Catheters of the present invention may also include sheaths and other types of tubular structures used for delivery of devices, instruments, or the like to target sites within the body.

As used herein, the term “proximal” refers to a direction toward an operator and the site of catheter introduction into a subject along the path of the catheter system, and “distal” refers to the direction away from the operator and introduction site along the path of the catheter system toward a terminal end of the catheter assembly.

Many different catheter types and constructions are known in the art and catheters of the present invention may have a variety of constructions, properties, and the like. Catheters may, for example, comprise a multi-layer construction in which flexible tubing is reinforced with stiffer materials such as helical coils and braided materials to provide different stiffness properties along the length of the catheter. Coatings may be provided on the inner or outer surfaces of catheters to improve lubricity. Hydrophilic coatings are often provided on exterior surfaces to facility guidance through tortuous vasculature. Liners comprising lubricious materials such as fluoropolymer resins, films and coatings, such as TEFLON® PTFE and similar materials may be provided on inner catheter surfaces to enhance passage of accessory devices and systems through the catheter.

Catheters may also have different cross-sectional dimensions and/or thicknesses and/or flexibilities along their lengths. In general, catheters have a larger cross-sectional outer dimension and have thicker, less flexible walls in proximal sections and a smaller cross-sectional outer dimension with thinner, more flexible walls in distal sections. The length of a guide catheter may be up to 100 cm or more, and the outer lumen diameter (French size) of a guide catheter may range from 4-10 F. The length of a microcatheter for use, for example, in neurovascular applications, may be up to 170-200 cm, and the outer lumen diameter of a microcatheter may range from about 1.5-3.5 F. Microcatheters often incorporate a flexible distal tip for a length of from about 5 cm to 50 cm from the terminal distal end that is soft, perhaps shapeable, and has one or more radiopaque markers for relative positioning by the physician. The tip configuration may be preformed or formable in a variety of configurations, including linear and curved, as well as angled. Catheters of the present invention may incorporate any of these features. U.S. Pat. Nos. 6,672,338, 6,152,944, 6,824,553, 6,863,678, 6,740,073, 6,626,889 and 6,679,836 are incorporated by reference herein in their entireties and disclose exemplary types of catheters and catheter constructions, any of which may be used in connection with catheters of the present invention. Numerous other catheter types and constructions are known in the art and may be used in combination with the novel catheter features described herein.

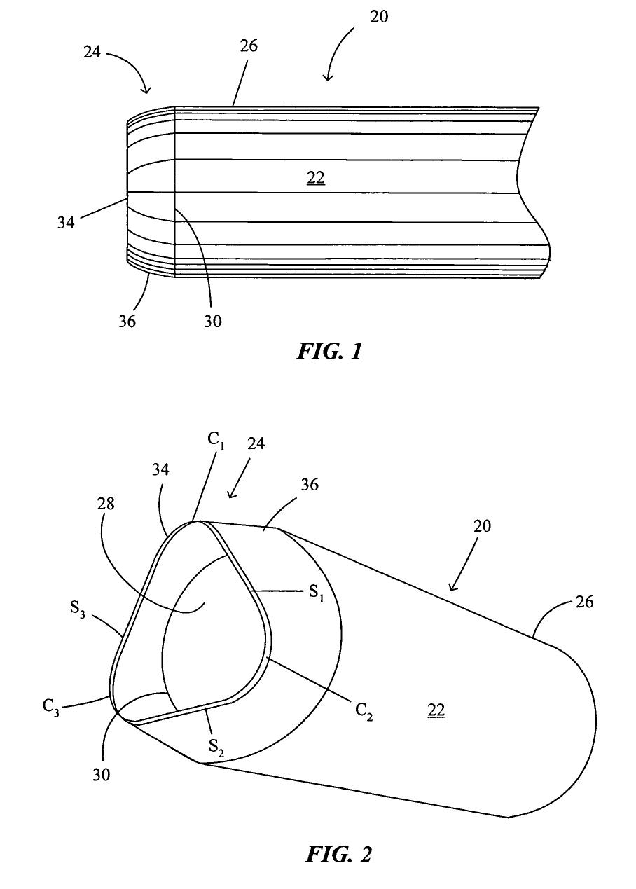

Catheters of the present invention comprise an elongate tubular member defining an inner lumen extending from a proximal end to a distal end of the tubular member. One aspect of catheters of the present invention is directed to the distal portion of the catheter and is illustrated schematically in FIGS. 1-5B, in which corresponding reference numerals are used to describe corresponding elements. The figures are schematic and are intended to illustrate applicants' catheter improvements rather than various structural details of the catheters. The distal catheter portion 20 comprises a generally tubular section 22 and a tapered section 24. Tapered section 24 has a discrete and more pronounced taper than the portion of generally tubular section 22 in proximity to the tapered section. Generally tubular section 22 has a substantially cylindrical outer wall 26 and a substantially cylindrical inner wall 28, forming the catheter lumen. The cross-sectional configuration of inner wall 28 in proximity to tapered section 24 is generally circular, as shown at distal cylindrical profile 30.

Tapered tip section 24 terminates in a distal surface 34 and comprises a tapered side wall 36 that may be angled or curved relative to the longitudinal axis of tubular section 22. In the embodiment shown in FIG. 1, side wall 36 tapers along a substantially curved line with respect to side wall 26. The degree of taper in this context is described as an angle drawn between a central longitudinal axis of the generally tubular section and a line joining a point on the distal cylindrical profile 30 and a corresponding point on distal surface 34. Taper angles of from about 0.05° to about 50° are suitable for use in distal catheter portions of the present invention. In general, a shallow taper angle of from about 0.05° to about 20° is used if the tapered tip section 24 is relatively long, having a side wall length of about 1 cm or more. A steeper taper angle of from about 5° to about 50° is generally preferred if the tapered tip section 24 is relatively short, having a side wall length of about 2 cm or less. Tapered tip section 24 preferably has a length of from about 1 mm to about 3 cm and, in one embodiment, is less than 1 cm in length.

In the catheter embodiments illustrated in FIGS. 1-4, the alignment and central longitudinal axes of tubular section 22 and tapered tip section 24 are substantially identical and generally correspond to the central longitudinal axis of the proximal and middle portions of the catheter. In alternative embodiments, the alignment and central longitudinal axes of distal catheter section 20 may be generally angled or curved, and the alignment of tapered tip section 24 may not correspond with that of tubular section 22.

In one embodiment, the cross-sectional configuration of the outer perimeter of tapered tip section 24 is generally circular and is substantially similar to, though of smaller diameter than, the cross-sectional configuration of tubular section 22. In alternative embodiments, the cross-sectional configuration of the outer perimeter of tapered tip section 24 in its distal region is different from the cross-sectional configuration of distal cylindrical profile 30. In one embodiment, illustrated in FIG. 2, the distal end of tapered tip section 24 has a substantially triangular cross-sectional configuration comprising three generally linear side sections S₁, S₂ and S₃ joined by three curved corner sections C₁, C₂ and C₃ forming a generally equilateral triangle with arced corners. Although the side sections S₁, S₂ and S₃ are described as generally linear, it will be appreciated that the side sections may have a slightly curved profile and, in combination with the curved corner sections, still form a generally triangular configuration.

Catheter tapered tip section 24 thus tapers in a distal direction to provide an outer surface having a smaller perimeter at a distal section than at a proximal section and, in some embodiments, a different cross-sectional profile at a distal section than at a proximal section. In the embodiment illustrated in FIG. 1, the internal and external cross-sectional configurations of tapered tip section 24 are substantially similar and both the inner and outer perimeter dimensions of the distal end 34 are smaller than those of tubular section 22. The wall thickness of tapered tip section 24 is substantially constant at distal end 34 in this embodiment and may be equal to, greater than or less than the wall thickness of the distal catheter section 20. In some embodiments, the wall thickness of tapered tip section 24 varies over the length of the tapered section.

Tapered tip section 24 is preferably flexible and may have a relatively high degree of elasticity. For catheter applications in which devices or instruments having a profile larger than that of the inner diameter of catheter tip section 24 are being introduced and/or withdrawn, for example, catheter tip section 24 is preferably elastic and deformable to provide passage of such devices and instruments. In one embodiment, arced corners C₁, C₂ and C₃ may have different flexibility or elasticity properties than side walls S₁, S₂ and S₃ and arced corners C₁, C₂ and C₃ may have different wall thicknesses than side walls S₁, S₂ and S₃.

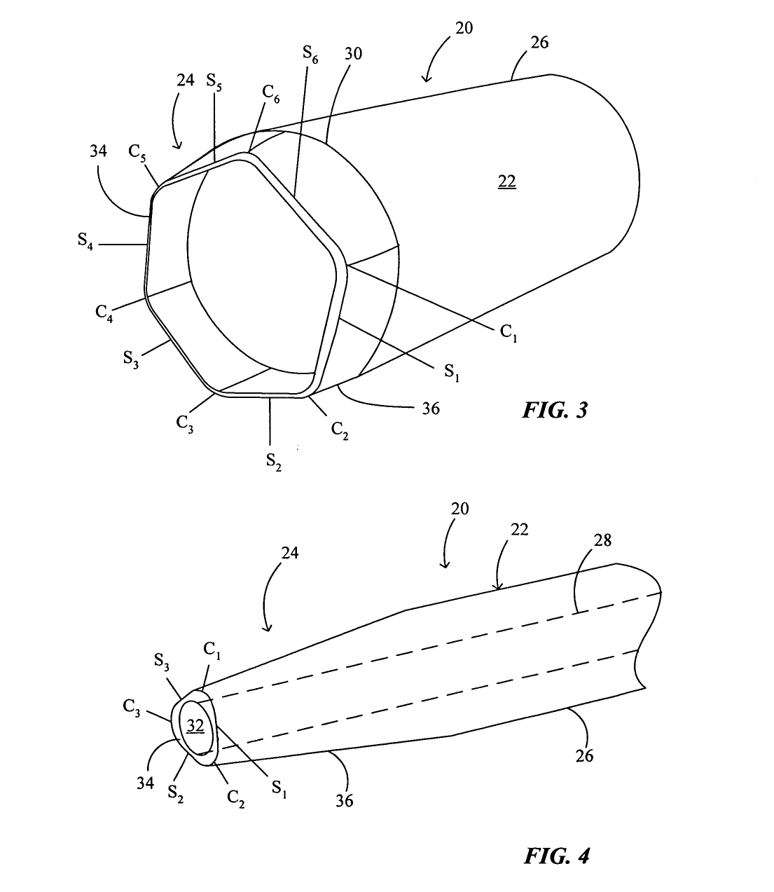

FIG. 3 illustrates another embodiment of distal catheter section 20 of the present invention in which tapered tip section 24 tapers in a distal direction and has an external surface having a smaller perimeter at a distal section than at a proximal section and a different cross-sectional profile at a distal section than at a proximal section. In this embodiment, the distal end 34 of tapered tip section 24 has a substantially hexagonal cross-sectional configuration comprising six generally linear side sections S₁-S₆ joined by six curved corner sections C₁-C₆. While distal catheter sections having generally triangular and generally hexagonal configurations are illustrated and described in detail, it will be appreciated that the cross-sectional configuration of distal end 34 may have a generally round or oval or oblong configuration, a triangular, square, pentagonal or hexagonal configuration, or any configuration having a number of arced corners joining sidewalls.

FIG. 4 illustrates another embodiment of a catheter distal tip section in which the cross-sectional dimension and profile of the inner catheter wall forming the catheter lumen remains substantially constant over the length of tubular section 22 and tapered tip section 24, while the cross-sectional dimension and profile of the outer catheter wall changes. In this embodiment, the inner catheter lumen in the distal region of the catheter has a constant configuration and dimension, preferably cylindrical, while the outer catheter wall tapers toward the distal terminal end and the generally cylindrical outer catheter wall proximal to the tapered distal end changes to a polygonal configuration, such as the generally triangular configuration illustrated. As shown in FIG. 4, the catheter lumen 32 at distal surface 34 is substantially cylindrical, while the outer catheter wall tapers and terminates in a substantially triangular configuration having arced corners C₁, C₂, C₃ and substantially linear side walls S₁, S₂, S₃.

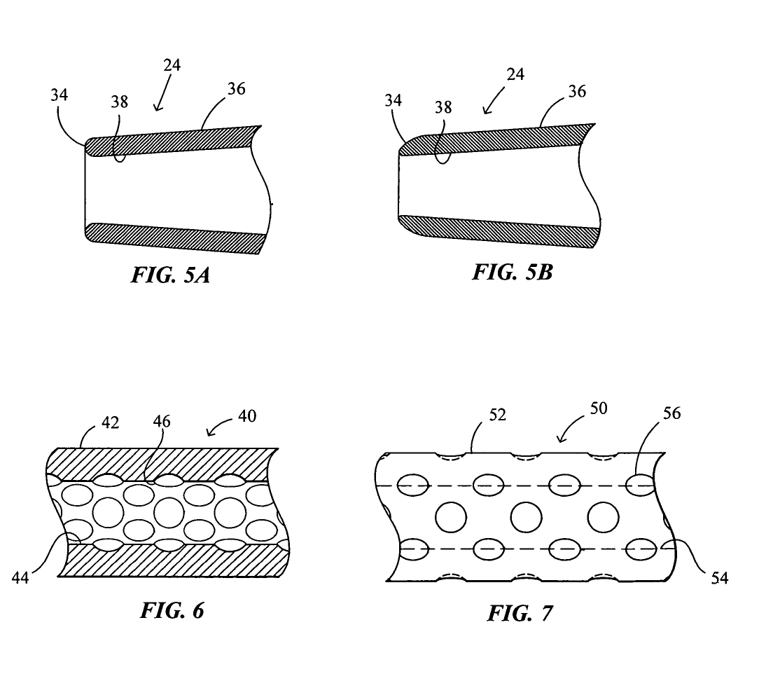

FIGS. 5A and 5B show cross-sectional views illustrating preferred profiles for distal surface 34 of tapered tip section 24. In the embodiment of FIG. 5A, distal surface 34 is substantially rounded in a semi-circular profile that joins outer distal surface 36 and inner lumen surface 38. In the embodiment of FIG. 5B, distal surface 34 is substantially rounded in a chamfered profile in which outer distal surface 36 curves toward and meets inner lumen surface 38. Both of these embodiments provide an atraumatic distal surface. Other atraumatic profiles may be used in connection with catheters of the present invention.

In another aspect, catheters of the present invention incorporate inner and/or outer catheter surfaces having a three-dimensional surface conformation. In one embodiment of a neurovascular microcatheter, for example, an inner catheter surface forming a lumen may be dimpled or grooved or provided with other types of surface discontinuities to reduce friction and enhance the slidability of and passage through the lumen of instruments, accessory devices, and the like. The outer surface of a catheter may also be provided with surface discontinuities to facilitate passage of the catheter through vessels and tortuous passageways.

FIG. 6 illustrates a catheter section 40 comprising an outer wall 42 having a substantially smooth surface and an inner lumen wall 44 having a plurality of surface discontinuities 46. FIG. 7 illustrates a catheter section 50 comprising an outer wall 52 having a plurality of surface discontinuities 56 and a generally smooth inner lumen surface 54. In the embodiments shown in FIGS. 5 and 6, surface discontinuities 46 and 56 are in the form of “dimples” or relatively shallow disc-shaped depressions. In alternative embodiments, the surface discontinuities may be generally triangular, oval, oblong, provided in curved arcs or serpentine shapes, or in any other configurations that facilitate movement of accessory devices or instruments trough an inner lumen or that facilitate translation of a catheter along its path. Although FIGS. 6 and 7 illustrate surface discontinuities provided on generally cylindrical catheter sidewalls, it will be recognized that such surface discontinuities may additionally or alternatively be provided in connection with tapered distal catheter tip sections or in connection with other tapered or shaped catheter sections.

Discontinuities 46 and 56 may be provided on inner and outer catheter walls, respectively, in areas where contact of the lumen wall with accessory devices or instruments reduces friction and enhances slidability. In preferred embodiments, the maximum depth of discontinuities 46 and 56 is relatively shallow and is less than about 6μ, while the maximum dimension of discontinuities 46 and 56, which is the diameter in the embodiment shown, is less than about 10μ and, more preferably, less than about 8μ. The pattern of surface discontinuities is preferably regular, though irregular patterns may be implemented for specific applications. The density and/or pattern and or configuration of discontinuities may vary along the length of a catheter or catheter section, with higher density discontinuities in narrower catheter sections to improve sliding of devices and instruments in the catheter lumen and/or sliding of the catheter outer surface within a vessel.

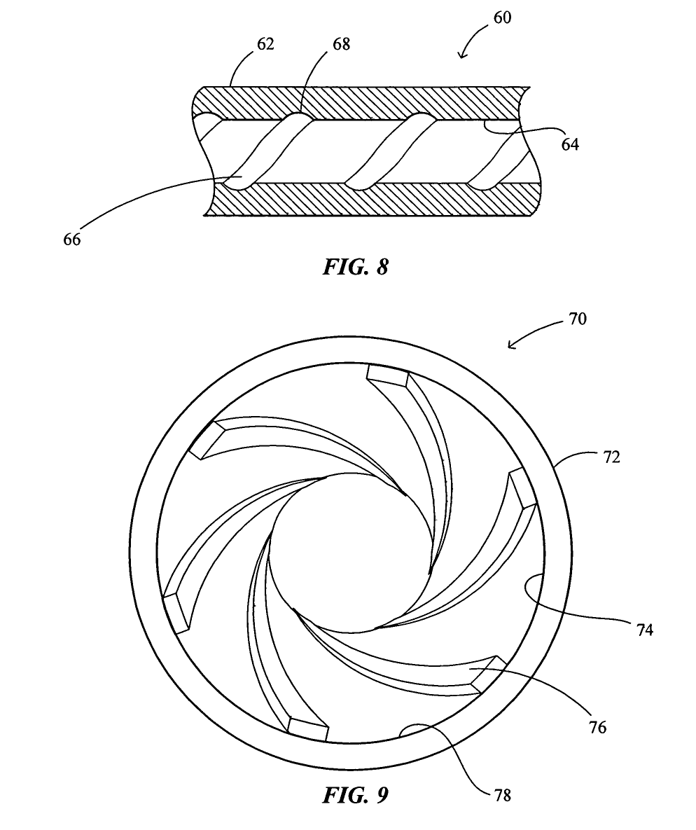

In yet another embodiment, an inner or outer catheter wall may be provided with surface discontinuities in the form of grooves that are generally longitudinal or curved or provided in a helical or spiral configuration. FIG. 8 illustrates a catheter or catheter section 60 having a generally cylindrical outer wall 62. Generally cylindrical lumen wall 64 has surface discontinuities along at least a portion of its length in the form of a helical groove 66 forming depressions 68 in lumen wall 64. Helical groove 66 may have a constant or variable pitch and may spiral in either a left or right direction, or may comprise sections spiraling in both left and right directions. Depressions 68 formed by helical groove 66 may have a curved profile, as shown, or they may have a generally rectilinear profile.

The grooved inner lumen wall of the catheter may alternatively or additionally comprise lands and grooves in a pattern that facilitates both passage and rotation of an accessory device or instrument through the lumen. These surface discontinuities may be provided in any region of a catheter and may, in some embodiments, be provided on the inner lumen wall of a distal tapered section of a catheter.

FIG. 9 illustrates a catheter or catheter section 70 having a generally cylindrical outer wall 72. Generally cylindrical lumen wall 74 has surface discontinuities along at least a portion of its length in the form of lands or projections 76 arranged on a curved or angled axis. The provision of lands 76 effectively forms grooves 78 between neighboring lands 76. The walls of lands 76 and grooves 78 may have a curved profile or may have a generally rectilinear profile, as shown. The projecting lands 76, in combination, preferably comprise less than 50% of the surface area of lumen wall 74 and, in some embodiments, comprise less than 20% or less than 15% of the surface area of lumen wall 74. Grooves 78 preferably comprise more than 50% of the surface area of lumen wall 74 and, in some embodiments, comprise more than 80%, or more than 85%, of the surface area of lumen wall 74.

The lands and grooves in this embodiment may be slightly curved or generally rectilinear, having generally flat or curved side walls. This pattern of lands and grooves is similar to rifling patterns used in rifle barrels to facilitate the transit and rotation of bullets or other projectiles. In a similar fashion, broach or button rifling techniques may be used to form surface discontinuities in connection with catheter or catheter sections of the present invention. Conventional rifling patterns that are suitable for use in catheters of the present invention include 4/right, 5/right, 6/right, 6/left, 8/right and 16/right.

While in the foregoing specification this invention has been described in relation to certain preferred embodiments thereof, and many details have been set forth for purposes of illustration, it will be apparent to those skilled in the art that the invention is susceptible to various changes and modification as well as additional embodiments and that certain of the details described herein may be varied considerably without departing from the basic spirit and scope of the invention.

All of the patent references and publications cited in this specification are incorporated by reference herein in their entireties.

We claim:

1. A catheter comprising a generally tubular structure having a substantially continuous side wall forming an outer catheter wall and an inner catheter lumen and having a tapered distal tip section terminating in a distal surface, wherein the tapered distal tip section has a tapered outer catheter wall profile in which the cross-sectional perimeter of the outer catheter wall at the distal surface is less than the cross-sectional perimeter of the outer catheter wall proximal to the distal surface and the cross-sectional profile of the outer catheter wall at the distal surface is different from the cross-sectional profile of the outer catheter wall proximal to the distal surface.

2. A catheter of claim 1, wherein the cross-sectional profile of the outer catheter wall at the distal surface is substantially triangular.

3. A catheter of claim 2, wherein the outer surface conformation of the distal section has arced corners separated by generally linear side walls.

4. A catheter of claim 3, wherein the arced corners have different flexibility or elasticity properties than the generally linear side walls.

5. A catheter of claim 1, wherein the cross-sectional profile of the catheter lumen at the distal surface substantially matches the cross-sectional profile of the outer catheter wall at the distal surface.

6. A catheter of claim 1, wherein the cross-sectional profile of the catheter lumen at the distal surface is different from the cross-sectional profile of the outer catheter wall at the distal surface.

7. A catheter of claim 6, wherein the cross-sectional profile of the catheter lumen at the distal surface is substantially circular.

8. A catheter of claim 1, wherein the distal surface of the tapered distal tip section is chamfered or rounded.

9. A catheter of claim 1, wherein the tapered distal tip section has a longitudinal axis that is tapered or curved relative to the longitudinal axis of the catheter proximal to the tapered distal tip section.

10. A catheter of claim 1, wherein the tapered distal tip section has a longitudinal axis that is aligned and coaxial with the longitudinal axis of the catheter proximal to the tapered distal tip section.

11. A catheter comprising a generally tubular structure having a substantially continuous side wall forming an outer catheter wall and an inner catheter lumen, wherein at least a portion of a surface of the inner catheter lumen has a three-dimensional surface conformation.

12. A catheter of claim 11, wherein at least a portion of a surface of the inner catheter lumen is dimpled.

13. A catheter of claim 11, wherein at least a portion of a surface of the inner catheter lumen has at least one groove.

14. A catheter of claim 13 comprising a plurality of grooves separated by projecting lands, wherein the grooves and lands follow a substantially curved path.

15. A catheter of claim 14, wherein the grooves

16. A catheter of claim 13, wherein the at least one groove follows a substantially helical path and has a curved profile.

17. A catheter of claim 15, wherein the at least one groove has a substantially spiral configuration.

18. A catheter of claim 11, wherein at least a portion of the outer catheter wall has a three-dimensional surface conformation.

19. A catheter of claim 17, wherein at least a portion of the outer catheter wall is dimpled.

20. A catheter of claim 11, having dimensions suitable for use in neurovascular vessels.
